(19) United States
(12) Patent Application Publication
(10) Pub. No.: US 2015/0246066 A1
(43) Pub. Date: Sep. 3, 2015

(54) NUTRITIONAL SUPPLEMENT

(71) Applicant: Daniel NELSON, Littlehampton (GB)

(72) Inventor: Daniel NELSON, Littlehampton (GB)

(21) Appl. No.: 14/634,240

(22) Filed: Feb. 27, 2015

(30) Foreign Application Priority Data
Feb. 28, 2014 (GB) 1403528.1

(51) Int. Cl. A61K 31/7004; A61K 31/675; A61K 31/155; A23L 1/302; A61K 31/194; A61K 9/00; A23L 1/305; A61K 31/198; A61K 31/20

(52) U.S. Cl. CPC A61K 31/7004; A61K 31/198; A61K 31/675; A61K 31/155; A61K 31/20; A61K 31/194; A61K 9/0056; A23L 1/3051; A23L 1/302

(57) ABSTRACT

A solid nutritional supplement formulation comprises at least 60 wt % dextrose combined with creatine and/or Branched Chain Amino Acids (BCAA's).

### CROSS REFERENCE TO RELATED APPLICATIONS

This Nonprovisional application claims priority under 35 U.S.C. §119(a) on Patent Application No. GB 1403528.1 filed in the United Kingdom on Feb. 28, 2014, the entire contents of which are hereby incorporated by reference.

### TECHNICAL FIELD

The invention relates to nutritional supplements, and particularly, although not exclusively, to nutritional supplement formulations which can be taken following exercise, or when a subject is in need of a quick and immediate boost in various nutrients, such as sugars, amino acids and vitamins. The invention is concerned with post-workout/exercise nutrition and supplementation.

### BACKGROUND

It is common knowledge that partaking in physical exercise provides the participant with a myriad of benefits. Should it be for weight control, increased performance in a particular sport, mood elevation, improved energy and cardiovascular health or purely for aesthetic reasons, millions of people exercise on a regular basis, and in order to continue performing optimally, and realise the full gains as a result of this exercise, arguably a key component is proper nutrition, in particular macronutrient ingestion and timing.

It is widely recognised that an individual's metabolism post exercise has increased nutrient sensitivity and this is a crucial period in which to reduce muscle breakdown (catabolism), lower cortisol (stress hormone) levels, initiate protein synthesis (anabolism), reduce the effects of DOMS (delayed onset muscle soreness) and improve future athletic performance. This period is more commonly referred to as the ‘Anabolic Window’ and is generally noted to last for a period of approximately 30 minutes post exercise.

During any intense physical activity ATP (adenosine triphosphate), creatine phosphate and glycogen (the storage form of glucose) reserves will be used and\or depleted and typically, the primary concern for an individual who has exercised, will be to replenish these stores in order to avoid muscle protein breakdown and to signal the body to begin muscle protein synthesis.

The fastest way to prevent this muscle breakdown, and replenish glycogen stores is to consume high GI (glycemic index) carbohydrates in order to raise blood sugar (glucose) levels and trigger an insulin release that drives glucose back into the cells via a process known as glycogenesis.

Therefore, a problem occurs due to standard post workout routines consisting of some or all of the following tasks: warm down, shower, clothing change, travel back to home/work. All of these tasks take time and all usually must occur before preparing/cooking a meal for consumption, thus reducing the athlete's potential to maximise gains and take advantage of the ‘anabolic window’. So the ability to have a high GI carbohydrate source, at the ready and stored in a gym bag, locker or car, that can be easily and readily consumed, has obvious benefits.

Other important nutrients that are commonly consumed post-exercise are Branched Chain Amino Acids (BCAA's), which are made up of three of the nine essential amino acids, which means that they cannot be synthesized by the body and therefore can only be obtained by the food we eat. BCAA's play an important role in building and maintaining muscle during and after exercise as they stimulate the pathways involved in muscle development as well as reduce exercise-induced muscle pain and decrease muscle fatigue. Additionally, their presence can help preserve muscle tissue and reduce muscle breakdown, so it is obvious why BCAA's are a vital post-workout ingredient.

Creatine may be the single most studied product in the history of dietary supplements. As well as providing additional energy for muscles (ATP) during intense exercise, creatine also appears to suppress muscle degradation, boost muscle protein synthesis and enhance stem cell proliferation that in turn supports new muscle fiber formation. The result is enhanced muscle recovery following training. It is also worth noting that several studies have been performed, and proven, that creatine, consumed with a high GI carbohydrate, such as dextrose, increases the rate of absorption and uptake into the body's cells.

Glutamine is the most abundant amino acid in your body. It accounts for more than 60% of the free amino acids in skeletal muscle and more than 20% of total circulating amino acids. This means that glutamine is especially concentrated in muscle. During intense training, your body can even lose up to 50% of its glutamine stores to stress and fatigue. It is for this reason, and due to its proven anti-catabolic properties, that glutamine is present in many recovery supplements.

Protein metabolism, growth and carbohydrate utilization are all made possible in part by the presence of vitamin B6. Studies on vitamin B6 in athletic performance show a definite increased need for athletes and possible performance enhancement from supplementation. Vitamin B6 also helps produce insulin and various neurotransmitters and enzymes in the body, making its role in physical growth and recovery extra important.

There is therefore a need for a nutritional supplement formulation that combines some, or all, of these nutritional elements in a convenient and easy to use form.

### BRIEF SUMMARY OF THE INVENTION

Accordingly, in a first aspect of the invention, there is provided a solid nutritional supplement formulation comprising at least 60 wt % dextrose combined with creatine and/or Branched Chain Amino Acids (BCAA's).

Advantageously, the solid nutritional supplement formulation of the invention combines several nutritional elements and solves the problems of convenience, portability, storage, taste, correct nutrient composition, bioavailability and durability that are not addressed by other currently available post workout products. Thus, the formulation provides a delivery system that offers a fast and highly convenient means for an exercised individual to ingest the necessary nutrients, in their correct ratios, in order to help realise the maximum benefits of their chosen activity and reduce the recovery time required for future optimal performance. The solid formulation can be readily carried in a gym bag or pocket, and can therefore be consumed immediately after a workout while the subject warms down, showers, changes their clothing and/or travels back to home or work. Accordingly, the usual delay in post workout nutritional supplementation with liquid drinks or shakes is significantly minimised or even avoided.

In one preferred embodiment, the nutritional supplement formulation is in the shape of a consumable sweet or lozenge (or ‘gobstopper’). The average dimensions of the sweet or lozenge are between 2 cm and 4 cm wide by 1.5 cm to 3 cm long.

In another preferred embodiment, the nutritional supplement formulation is in the shape of a tablet. The average dimensions of this embodiment would be approximately 0.5 cm to 1 cm long, and between 1 cm and 2 cm in diameter for a cylindrical style tablet, and between 2 cm and 3 cm long by 1 cm to 1.5 cm wide for a more rectangular style tablet.

In a most preferred embodiment, however, the nutritional supplement formulation is in the form of a lollipop comprising a solid head attached to a protruding stick. The formulation comprising the dextrose combined with creatine and/or BCAA's is shaped to form the head from which a stick extends, and by which a user can hold the formulation. The average dimensions of this embodiment are between 2 cm and 4 cm in diameter by 1.5 cm to 3 cm long.

Advantageously, a lollipop is an immediate and continued source of nutrients, specifically designed for recovery purposes, into the consumer's digestive system. In its lollipop form, the formulation is sucked, chewed and swallowed by the consumer until only the stick, which is not consumable, remains. The form of the lollipop allows for a convenient and simple method of administration for the ingredients, in a single dose. It also enables the nutritional components of the formulation, in particular the dextrose, to take advantage of trans-mucosal absorption, thus increasing the speed and pathways utilised to enter into the bloodstream.

Preferably, the supplement formulation comprises at least 70% dextrose, more preferably at least 80% dextrose, even more preferably at least 85 wt % dextrose, and more preferably at least 90% dextrose.

Preferably, the supplement formulation comprises 1-25 wt % creatine, more preferably 2-15 wt % creatine, and most preferably 4-6 wt % creatine.

Preferably, the supplement formulation comprises 1-25 wt % BCAA's, more preferably 2-15 wt % BCAA's, and most preferably 4-6 wt % BCAA's. Preferably, the BCAA's comprise leucine, isoleucine and/or valine, and more preferably at least two amino acids, and most preferably all three amino acids.

In preferred embodiments, the formulation can also include a further ingredient selected from a group of ingredients consisting of: glutamine; vitamin B6; magnesium stearate; citric acid; flavourant; sweeteners (preferably, sucralose).

Preferably, the supplement formulation comprises 1-10 wt % glutamine, more preferably 2-8 wt % glutamine, and most preferably 4-6 wt % glutamine.

Preferably, the supplement formulation comprises 0.1-0.5 wt % vitamin B6, more preferably 0.15-0.35 wt % vitamin B6, and most preferably 0.2-0.3 wt % vitamin B6.

Preferably, the supplement formulation comprises 1-8 wt % magnesium stearate, more preferably 2-6 wt % magnesium stearate, and most preferably 3-4 wt % magnesium stearate.

Preferably, the supplement formulation comprises 0.5-3 wt % citric acid more preferably 0.75-2 wt % citric acid, and most preferably 1-1.5 wt % citric acid.

Preferably, the supplement formulation comprises 0.1-1 wt % flavourant, more preferably 0.2-0.7 wt % flavourant, and most preferably 0.3-0.5 wt % flavourant.

Preferably, the supplement formulation comprises 0.01-0.1 wt % sweetener, more preferably 0.02-0.08 wt % sweetener, and most preferably 0.03-0.06 wt % sweetener.

Typically, the nutritional supplement formulation is 2 g-30 g in weight, preferably 10-20 g in weight.

Hence, in one preferred embodiment, the nutritional supplement formulation comprises 4000 mg-25000 mg Dextrose and 250 mg-2000 mg creatine.

In another preferred embodiment, the nutritional supplement formulation comprises 4000 mg-25000 mg Dextrose and 200 mg-3000 mg BCAA's.

In another preferred embodiment, the nutritional supplement comprises 4000 mg-25000 mg Dextrose, 250 mg-2000 mg creatine and 200 mg-3000 mg BCAA's.

In another embodiment, the formulation may further comprise glutamine, preferably 250 mg-2000 mg Glutamine. Thus, in another preferred embodiment, the nutritional supplement formulation comprises 4000 mg-25000 mg Dextrose, 250 mg-2000 mg creatine 200 mg-3000 mg BCAA's, and 250 mg-2000 mg Glutamine.

In other embodiments, the formulation may further comprise vitamin B6, preferably 2 mg-72 mg Vitamin B6. Thus, in another preferred embodiment, the nutritional supplement formulation comprises 4000 mg-25000 mg Dextrose, 250 mg-2000 mg creatine 200 mg-3000 mg BCAA's, 250 mg-2000 mg Glutamine, and 2 mg-72 mg Vitamin B6.

In other embodiments, the formulation may further comprise Magnesium Stearate, preferably 100-1000 mg Magnesium Stearate. Thus, in another preferred embodiment, the nutritional supplement formulation comprises 4000 mg-25000 mg Dextrose, 250 mg-2000 mg creatine 200 mg-3000 mg BCAA's, 250 mg-2000 mg Glutamine, 2 mg-72 mg Vitamin B6, and 100-1000 mg Magnesium Stearate. In other embodiments, the formulation may further comprise Citric Acid, preferably 50 mg-500 mg Citric Acid. Thus, in another preferred embodiment, the nutritional supplement formulation comprises 4000 mg-25000 mg Dextrose, 250 mg-2000 mg creatine 200 mg-3000 mg BCAA's, 250 mg-2000 mg Glutamine, 2 mg-72 mg Vitamin B6, 100-1000 mg Magnesium Stearate, and 50 mg-500 mg Citric Acid.

In other embodiments, the formulation may further comprise a Flavouring agent, preferably 10 mg-20 mg Flavouring agent. Thus, in another preferred embodiment, the nutritional supplement formulation comprises 4000 mg-25000 mg Dextrose, 250 mg-2000 mg creatine 200 mg-3000 mg BCAA's, 250 mg-2000 mg Glutamine, 2 mg-72 mg Vitamin B6, 100-1000 mg Magnesium Stearate, 50 mg-500 mg Citric Acid, and 10 mg-20 mg Flavouring agent.

In other embodiments, the formulation may further comprise a sweetener, preferably 2 mg-30 mg sweetener. Preferably, the sweetener is sucralose. Thus, in another preferred embodiment, the nutritional supplement formulation comprises 4000 mg-25000 mg Dextrose, 250 mg-2000 mg creatine 200 mg-3000 mg BCAA's, 250 mg-2000 mg Glutamine, 2 mg-72 mg Vitamin B6, 100-1000 mg Magnesium Stearate, 50 mg-500 mg Citric Acid, 10 mg-20 mg Flavouring agent, and 2 mg-30 mg Sucralose.

The inventor believes that the nutritional supplement formulation of the invention exhibits therapeutic efficacy by providing vital nutrients to a subject.

As such, in a second aspect of the invention, there is provided the nutritional supplement formulation according to the first aspect, for use in therapy or as a medicament.

In a third aspect of the invention, there is provided a nutritional supplement formulation according to the first aspect, for use in reducing the recovery time of a subject following exercise.

Advantageously, the formulation reduces the time required for the subject to attain future optimal performance following a workout (i.e. post workout recovery).

In a fourth aspect of the invention, there is provided a method for reducing the recovery time of a subject following exercise, the method comprising administering, to a subject in need of such treatment, the nutritional supplement formulation according to the first aspect.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

### BRIEF DESCRIPTION OF THE DRAWINGS

FIG. 1 is a schematic side view of one embodiment of the nutritional supplement formulation of the invention. As can be seen the supplement is in the form of a compressed formulation lollipop in which the lollipop ‘head’ can clearly be seen attached to a standard lollipop stick;

FIG. 2 shows a plan view of the nutritional supplement shown in FIG. 1 from above;

FIG. 3 shows a side view of a second embodiment of the nutritional supplement (i.e. lozenge or gobstopper) in which the lollipop head has been produced without a stick; and

FIG. 4 shows a perspective view of a third embodiment (i.e. tablet) of the nutritional supplement. This embodiment is a smaller confectionary style format.

### DETAILED DESCRIPTION OF THE INVENTION

### Examples

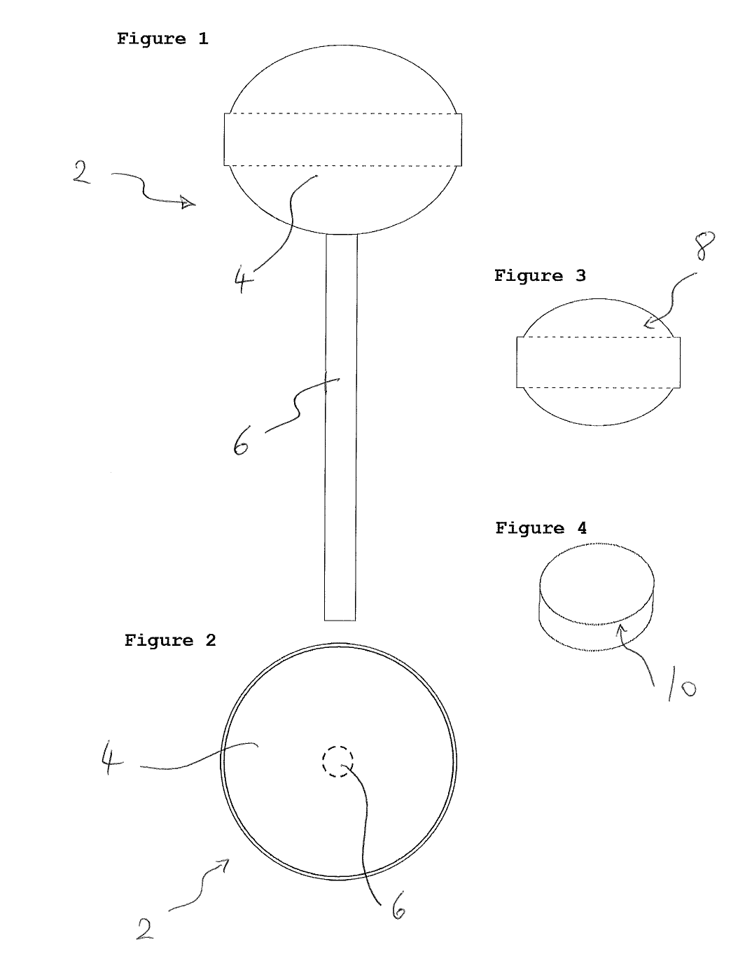

Referring to FIGS. 1 and 2, there is shown a first embodiment of a nutritional supplement formulation 2 in the shape of a lollipop. The lollipop 2 has a compressed edible head 4 and a non-edible stick 6 onto which a user grips. The head 4 contains the active ingredients making up the supplement formulation. The dimensions of the head 4 are 3 cm in diameter by 2.5 cm long or tall, and the stick 6 is about 10 cm long.

Referring to FIG. 3, there is shown a second embodiment of the formulation 8, i.e. a gobstopper or lozenge. The entire lozenge 8 is made up of the active ingredients making up the supplement formulation. The dimensions of the lozenge 8 are 2.5 cm in diameter by 2 cm tall.

Finally, referring to FIG. 4, there is shown a third embodiment of the formulation 10, i.e. a sweet. The sweet 10 is smaller than the lozenge 8, and is about 1.5 cm in diameter by 0.5 cm tall.

In each embodiment described herein, the active agents of the nutritional formulation are (i) dextrose, and either (ii) BCAA's or (iii) creatine, or both. These two or three active agents, which in other embodiments can be combined with other ingredients, are dispensed, sized (milled, crushed, grinded and pulverised), and blended. The formulation then undergoes granulation, to provide homogeneity of the ingredients in the blend, before being dried then inserted into a mould or die (not shown), and then compressed under high pressure, by hardened steel punches, in order to produce a solid tablet (i.e. embodiments 8, 10) or lollipop head 4 (embodiment 2). For the lollipop embodiment, the stick 6 is inserted into the mix of ingredients while in the mould prior to compression, or it can be introduced after compression. The stick 6 is provided for ease of holding during use. The formulation can then be consumed as soon as possible following an intense exercise session, or at times when an individual is in need for an immediate boost in nutritional supplementation.

In some embodiments, the homogeneous formulation (i.e. excluding the stick 6) can be between 2 g-30 g in weight. Typically, however, the compressed, active ingredient in the formulation is approximately 15 g in weight. The formulation typically comprises: 80-85 wt % Dextrose, 5 wt % Creatine and 5 wt % BCAA's (in a 2:1:1 split—2.5% Leucine, 1.25% Isoleucine, 1.25% Valine).

Accordingly, a typical example of the basic formulation (weighing 15,000 mg) is provided in the following table:

| Ingredient | In Formulation (mg) | Acceptable Ranges (mg) |
|---|---|---|
| Dextrose | 12040.5 | 4000-25000 |
| Creatine | 750 | 250-3000 |
| Leucine (in BCAA) | 375 | 100-2000 |
| Isoleucine (in BCAA) | 187.5 | 50-1000 |
| Valine (in BCAA) | 187.5 | 50-1000 |

In some embodiments, the formulation can also comprise one or more ingredients selected from: Glutamine (5 wt %), Vitamin B6 (0.25 wt %), Magnesium Stearate (3 wt %) and/or Citric Acid (1 wt %), as well as flavourings (0.4 wt %) and/or sweetener (0.08 wt %) (e.g. sucralose). Examples of additional components of the formulation (weighing 15,000 mg) include:

| Ingredient | In Formulation (mg) | Acceptable Ranges (mg) |
|---|---|---|
| Glutamine | 750 | 250-2000 |
| Vitamin B6 | 37.5 | 2-75 |
| Magnesium Stearate | 450 | 100-1000 |
| Citric acid | 150 | 50-500 |
| Flavouring | 60 | 10-200 |
| Sucralose | 12 | 2-30 |

Thus, one example of the constituents of a preferred nutritional formulation (weighing 15,000 mg) includes:

| Ingredient | In Formulation (mg) | Acceptable Ranges (mg) |
|---|---|---|
| Dextrose | 12040.5 | 4000-25000 |
| Creatine | 750 | 250-3000 |
| Glutamine | 750 | 250-2000 |
| BCAA | 750 | 200-4000 |
| Vitamin B6 | 37.5 | 2-75 |

Another example of the constituents of a preferred nutritional formulation (weighing 15,000 mg) includes:

| Ingredient | In Formulation (mg) | Acceptable Ranges (mg) |
|---|---|---|
| Dextrose | 12040.5 | 4000-25000 |
| Creatine | 750 | 250-3000 |
| Glutamine | 750 | 250-2000 |
| BCAA | 750 | 200-4000 |
| Vitamin B6 | 37.5 | 2-75 |
| Magnesium stearate | 450 | 100-1000 |

Another example of the constituents of a preferred nutritional formulation (weighing 15,000 mg) includes:

| Ingredient | In Formulation (mg) | Acceptable Ranges (mg) |
|---|---|---|
| Dextrose | 12040.5 | 4000-25000 |
| Creatine | 750 | 250-3000 |
| Glutamine | 750 | 250-2000 |
| BCAA | 750 | 200-4000 |
| Vitamin B6 | 37.5 | 2-75 |
| Magnesium stearate | 450 | 100-1000 |
| Citric acid | 150 | 50-500 |

Another example of the constituents of a preferred nutritional formulation (weighing 15,000 mg) includes:

| Ingredient | In Formulation (mg) | Acceptable Ranges (mg) |
|---|---|---|
| Dextrose | 12040.5 | 4000-25000 |
| Creatine | 750 | 250-3000 |
| Glutamine | 750 | 250-2000 |
| BCAA | 750 | 200-4000 |
| Vitamin B6 | 37.5 | 2-75 |
| Magnesium stearate | 450 | 100-1000 |
| Citric acid | 150 | 50-500 |
| Flavouring agent | 60 | 10-200 |

Another example of the constituents of a preferred nutritional formulation (weighing 15,000 mg) includes:

| Ingredient | In Formulation (mg) | Acceptable Ranges (mg) |
|---|---|---|
| Dextrose | 12040.5 | 4000-25000 |
| Creatine | 750 | 250-3000 |
| Glutamine | 750 | 250-2000 |
| BCAA | 750 | 200-4000 |
| Vitamin B6 | 37.5 | 2-75 |
| Magnesium stearate | 450 | 100-1000 |
| Citric acid | 150 | 50-500 |
| Flavouring agent | 60 | 10-200 |
| Sucralose | 12 | 2-30 |

Advantages of the nutritional supplement formulation reside in the way in which it combines several nutritional elements and elegantly solves the problems of convenience, portability, storage, taste, correct nutrient composition, bioavailability and durability that are not addressed by other currently available post workout products. The formulation provides a delivery system that offers a fast and highly convenient means for an exercised individual to ingest the necessary nutrients, in their correct ratios, in order to help realise the maximum benefits of their chosen activity and reduce the recovery time required for future optimal performance. The embodiment of the formulation which is a lollipop allows a user to hold the stick 6.

1. A solid nutritional supplement formulation comprising at least 60 wt % dextrose combined with creatine and/or Branched Chain Amino Acids (BCAA's).

2. A formulation according to claim 1, wherein the nutritional supplement formulation is in the shape of a consumable sweet, lozenge, or tablet.

3. A formulation according to claim 1, wherein the nutritional supplement formulation is in the form of a lollipop comprising a solid head attached to a protruding stick.

4. A formulation according to claim 1, wherein the supplement formulation comprises at least 70% dextrose or at least 80% dextrose.

5. A formulation according to claim 1, wherein the supplement formulation comprises at least 85 wt % dextrose or at least 90% dextrose.

6. A formulation according to claim 1, wherein the supplement formulation comprises 1-25 wt % creatine or 2-15 wt % creatine or 4-6 wt % creatine.

7. A formulation according to claim 1, wherein the supplement formulation comprises 1-25 wt % BCAA's or 2-15 wt % BCAA's or 4-6 wt % BCAA's.

8. A formulation according to claim 1, wherein the BCAA's comprise leucine, isoleucine and/or valine.

9. A formulation according to claim 1, wherein the formulation comprises a further ingredient selected from a group of ingredients consisting of: glutamine;
vitamin B6; magnesium stearate; citric acid; flavourant; and sweetener.

10. A formulation according to claim 1, wherein the supplement formulation comprises 1-10 wt % glutamine or 2-8 wt % glutamine or 4-6 wt % glutamine.

11. A formulation according to claim 1, wherein the supplement formulation comprises 0.1-0.5 wt % vitamin B6 or 0.15-0.35 wt % vitamin B6 or 0.2-0.3 wt % vitamin B6.

12. A formulation according to claim 1, wherein the supplement formulation comprises 1-8 wt % magnesium stearate or 2-6 wt % magnesium stearate or 3-4 wt % magnesium stearate.

13. A formulation according to claim 1, wherein the supplement formulation comprises 0.5-3 wt % citric acid or 0.75-2 wt % citric acid or 1-1.5 wt % citric acid.

14. A formulation according to claim 1, wherein the supplement formulation comprises 0.1-1 wt % flavourant or 0.2-0.7 wt % flavourant or 0.3-0.5 wt % flavourant.

15. A formulation according to claim 1, wherein the supplement formulation comprises 0.01-0.1 wt % sweetener or 0.02-0.08 wt % sweetener or 0.03-0.06 wt % sweetener.

16. A method for reducing the recovery time of a subject following exercise, the method comprising administering, to a subject in need of such treatment, the nutritional supplement formulation according to claim 1.

17. A method according to claim 16, wherein the nutritional supplement formulation is in the form of a lollipop comprising a solid head attached to a protruding stick.

18. A method according to claim 16, wherein the supplement formulation comprises at least 70 wt % dextrose, 1-25 wt % creatine, 1-25 wt % BCAA's, and leucine, isoleucine and/or valine.
